(19) United States
(12) Patent Application Publication
(10) Pub. No.: US 2024/0065888 A1
(43) Pub. Date: Feb. 29, 2024

(54) NOZZLE FOR AN OPHTHALMIC FLUID DELIVERY DEVICE

(71) Applicant: TWENTY TWENTY THERAPEUTICS LLC, South San Francisco, CA (US)

(72) Inventors: Maxim Latrouitte Armstrong, South San Francisco, CA (US); Timothy Stowe, Alameda, CA (US); Siva Udayamurthy, South San Francisco, CA (US)

(21) Appl. No.: 18/238,431

(22) Filed: Aug. 25, 2023

Related U.S. Application Data
(60) Provisional application No. 63/400,832, filed on Aug. 25, 2022.

(51) Int. Cl. A61F 9/00

(52) U.S. Cl. CPC A61F 9/0026

(57) ABSTRACT

A nozzle for a fluid delivery device includes a nozzle wall having opposing interior and exterior nozzle surfaces, and at least one opening through which fluid is configured to be selectively delivered to a target site of a user during use of the fluid delivery device. The at least one opening is defined by an inner opening surface and extends through the nozzle wall from the interior nozzle surface to the exterior nozzle surface. The exterior nozzle surface is configured to be directed toward the eye of the user during use of the fluid delivery device. The exterior nozzle surface is configured to be at least hydrophobic. At least a portion of the inner opening surface is configured to be hydrophilic.

### RELATED APPLICATION

This application claims priority from U.S. Provisional Application No. 63/400,832, filed 25 Aug. 2022, the subject matter of which is incorporated herein by reference in its entirety.

### TECHNICAL FIELD

This disclosure relates to nozzles for ophthalmic fluid delivery devices.

### BACKGROUND

Non-gravitational fluid delivery devices for the non-gravitational delivery of fluids (e.g., ophthalmic drugs and/or viscous ophthalmic drugs) to a portion of the user (e.g., to the user's eye(s), nose, and/or mouth) are known. For ophthalmic and other applications, such fluids are normally based upon aqueous formulations (i.e. ones which primarily constitute water but are isotonic with other bodily fluids such as tears). U.S. application Ser. No. 15/931,482 (“the '482 application”), filed 13 May 2020 by Stowe and titled “Non-Gravitational Fluid Delivery Device For Ophthalmic Applications”, the subject matter of which is incorporated by reference in its entirety, discloses non-gravitational fluid delivery devices. FIG. 1 illustrates an example fluid delivery device 100 from the '482 application. The fluid delivery device 100 includes an applicator 102 and a cartridge 104 that is removably positioned within the applicator 102.

FIG. 2 illustrates an example cartridge 104 from the '482 application. The cartridge 104 includes a housing 206 and a head 208 that is attached to the housing 206. The head 208 may optionally include a protective head cover 210. As shown in FIG. 3, the housing 206 is a fluid reservoir or forms a chamber 312 in which the fluid is accommodated.

The head 208 is coupled to the housing 206 to dispense the fluid from the chamber 312. Generally, the head 208 is at least temporarily in fluid communication with the chamber 312 and forms a nozzle 314 and an air entry port 316. The head 208 also includes a cap 318 and a wall 320 that are at least partially movable relative to the nozzle 314. The cap 318 stays in a closed position unless fluid is about to be or is being ejected from the nozzle 314 at which time the cap 318 transitions into an open position.

The head 208 forms a holding chamber 322 that is in fluid communication with the chamber 312 and that is positioned between the nozzle 314 and the wall 320. The wall 320 is a membrane or elastomeric wall that is “squeezable” or flexible enough to deform in response to a striking force being applied to the wall 320. When a force is applied on the wall 320, the wall 320 deforms towards the nozzle 314 thereby reducing the volume of the holding chamber 322 and forcing the fluid from the nozzle 314. Movement of the wall 320 to back to its natural state after being struck fills the holding chamber 322 with fluid from the chamber 312 to prepare for another ejection of fluid.

As shown in FIG. 4, the nozzle 314 may include two slit openings 424, 426 through which fluid is dispensed from the holding chamber 322. The openings 424, 426 extend through a nozzle wall 428 of the nozzle 314 from an interior nozzle surface 430 of the nozzle wall 428 to an exterior nozzle surface 432 of the nozzle wall 428. The nozzle-opening configuration shown in FIG. 4 is but one example of the nozzle-opening configurations illustrated and discussed in the '482 application. For example, the '482 application discusses that the nozzle 314 may have an array of openings, the two slit openings 424, 426, or a single opening, and also describes or depicts a variety of different shapes and sizes for the opening(s).

As illustrated in FIG. 4, the cap 318, when in the closed position, may abut a head engagement surface 434 of the head 208 that at least partially surrounds the nozzle 314. During such engagement, the cap 318 is spaced from the nozzle 314 to form a moisture chamber 436 between the exterior nozzle surface 432 and the cap 318. Spacing the cap 318 from the nozzle 314 in the closed position helps reduce the likelihood of contaminating the nozzle 314 with the cap 318 as the nozzle 314 is not directly touched by the cap 318. Even with this spacing, it is possible for excess fluid and/or outside contaminates (e.g., dust and/or other small particles) to collect on the exterior nozzle surface 432 of the wall 428 during use of the fluid delivery device 100.

### SUMMARY

In an aspect, alone or in combination with any other aspect, a nozzle for a fluid delivery device comprises a nozzle wall having opposing interior and exterior nozzle surfaces, and at least one opening through which fluid is configured to be selectively delivered to a target site of a user during use of the fluid delivery device. The at least one opening is defined by an inner opening surface and extends through the nozzle wall from the interior nozzle surface to the exterior nozzle surface. The exterior nozzle surface is configured to be directed toward the eye of the user during use of the fluid delivery device. The exterior nozzle surface is configured to be at least hydrophobic. At least a portion of the inner opening surface is configured to be hydrophilic.

In an aspect, alone or in combination with any other aspect, a nozzle for a fluid delivery device comprises a nozzle wall having opposing interior and exterior nozzle surfaces, and at least one opening through which fluid is configured to be selectively delivered to a target site of a user during use of the fluid delivery device. The at least one opening is defined by an inner opening surface and extends through the nozzle wall from the interior nozzle surface to the exterior nozzle surface. The exterior nozzle surface is configured to be directed toward the eye of the user during use of the fluid delivery device. The exterior nozzle surface is at least hydrophobic via at least one of: a micropattern on the exterior nozzle surface; a hydrophobic or superhydrophobic coating on the exterior nozzle surface; a material forming the exterior nozzle surface being naturally hydrophobic or superhydrophobic; a chemical modification of the exterior nozzle surface; and nanometer-sized features on the exterior nozzle surface. At least one of the interior nozzle surface and the inner opening surface is configured to be hydrophilic via at least one of: a hydrophilic coating on at least one of the interior nozzle surface and the inner opening surface; a material forming at least one of the interior nozzle surface and the inner opening surface being naturally hydrophilic; and a chemical modification of the exterior nozzle surface.

### BRIEF DESCRIPTION OF THE DRAWINGS

For a better understanding, reference may be made to the accompanying drawings, in which:

FIG. 1 is a perspective front view of a prior art non-gravitational fluid delivery device;

FIG. 2 is a perspective rear view of a component of the prior art non-gravitational fluid delivery device of FIG. 1;

FIG. 3 is a cross-sectional view of a portion of the component of FIG. 2;

FIG. 4 is a cross-sectional view of a portion of the component of FIG. 2;

FIG. 5 is a perspective front view of a nozzle according to one aspect of the present invention;

FIG. 6 is a cross-sectional view of the nozzle of FIG. 5;

FIG. 7 is a cross-sectional view of a portion of a fluid delivery device having the nozzle of FIG. 5;

FIG. 8 is a side view of a portion of the nozzle of FIG. 5, including a portion of the nozzle in an alternate configuration;

FIG. 9 is a perspective front view of the nozzle of FIG. 1, including a portion of the nozzle in an alternate configuration;

FIG. 10 is a cross-sectional view of the nozzle of FIG. 9; and

FIG. 11 is a cross-sectional view of the fluid delivery device, including a portion of the fluid delivery device in an alternate configuration.

### DESCRIPTION OF ASPECTS OF THE DISCLOSURE

As used herein, the term “user” can be used interchangeably to refer to an individual who prepares for, assists with, and/or performs the operation of a tool, and/or to an individual who prepares for, assists with, and/or performs a procedure.

As used herein, the singular forms “a,” “an” and “the” can include the plural forms as well, unless the context clearly indicates otherwise. It will be further understood that the terms “comprises” and/or “comprising,” as used herein, can specify the presence of stated features, steps, operations, elements, and/or components, but do not preclude the presence or addition of one or more other features, steps, operations, elements, components, and/or groups thereof.

As used herein, the term “and/or” can include any and all combinations of one or more of the associated listed items.

As used herein, phrases such as “between X and Y” can be interpreted to include X and Y.

As used herein, the phrase “at least one of X and Y” can be interpreted to include X, Y, or a combination of X and Y. For example, if an element is described as having at least one of X and Y, the element may, at a particular time, include X, Y, or a combination of X and Y, the selection of which could vary from time to time. In contrast, the phrase “at least one of X” can be interpreted to include one or more Xs.

It will be understood that when an element is referred to as being “on,” “attached” to, “coupled” with, etc., another element, it can be directly on, attached to or coupled with the other element or intervening elements may also be present. In contrast, when an element is referred to as being, for example, “directly touching” another element, there are no intervening elements present. It will also be appreciated by those of skill in the art that references to a structure or feature that is disposed “directly adjacent” another feature may have portions that overlap or underlie the adjacent feature, whereas a structure or feature that is disposed “adjacent” another feature may not have portions that overlap or underlie the adjacent feature.

It will be understood that, although the terms “first,” “second,” etc. may be used herein to describe various elements, these elements should not be limited by these terms. These terms are only used to distinguish one element from another. Thus, a “first” element discussed below could also be termed a “second” element without departing from the teachings of the present disclosure. The sequence of operations (or steps) is not limited to the order presented in the claims or Figures unless specifically indicated otherwise.

Throughout this disclosure, various aspects of this invention can be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from about 1 to about 6 should be considered to have specifically disclosed subranges such as from 1 to 3, from 1 to 4, from 1 to 5, from 2 to 4, from 2 to 6, from 3 to 6 etc., as well as individual and partial numbers within that range, for example, 1, 1.1, 2, 2.8, 3, 3.2, 4, 4.7, 4.9, 5, 5.5 and 6. This applies regardless of the breadth of the range.

The invention comprises, consists of, or consists essentially of the following features, in any combination.

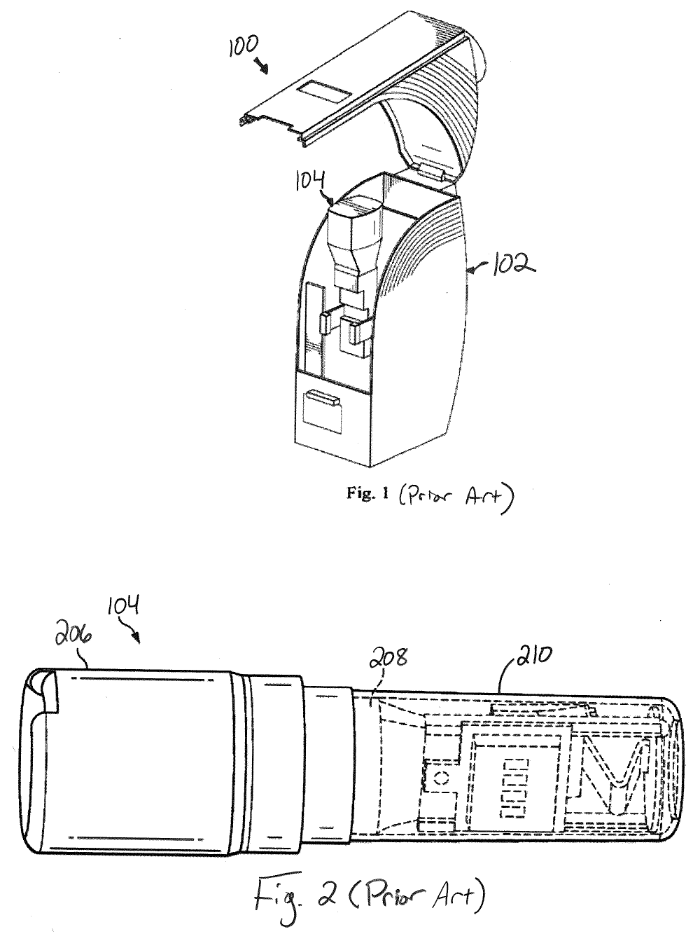

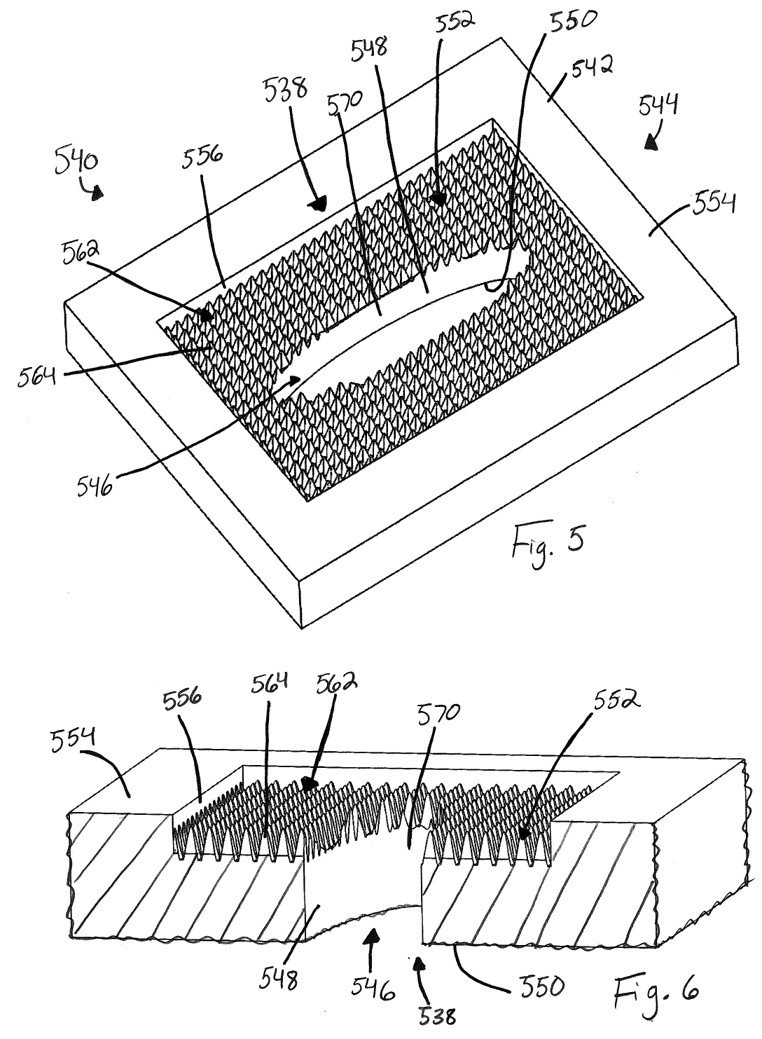

FIGS. 5-7 depict an example nozzle 538 designed in accordance with the present disclosure. The nozzle 538 and/or other teachings disclosed herein may be applicable to non-gravitational fluid delivery devices (e.g., the non-gravitational fluid delivery device 100 of FIGS. 1-4 and/or any non-gravitational fluid delivery device disclosed in the '482 application), gravitational fluid delivery devices (e.g., a standard eye dropper), and/or any other fluid delivery device. In the example configuration of FIGS. 5-6, the nozzle 538 is a part of a non-gravitational fluid delivery device 540 and is formed integrally with a head 542 of a cartridge 544. The non-gravitational fluid delivery device 540, the head 542, and the cartridge 544 may each substantially be, or may substantially be a modified version of, the non-gravitational fluid delivery device 100, the head 208, and the cartridge 104 as depicted in FIGS. 1-4.

Although the nozzle 538 is described as being formed integrally with the head 542, the nozzle 538 may be formed separately from the head 542 and then attached thereto.

As shown in FIGS. 5, the nozzle 538 includes an opening 546 through which fluid (e.g., an ophthalmic drug and/or a viscous ophthalmic drug) may be dispensed to an eye of a user. The fluid may be an ophthalmic fluid having an aqueous-based formulation. The nozzle 538, however, may be applicable to deliver a fluid to any desired target site of the user, such as to, for example, at least one of the user's nose, mouth, ear(s), limb(s), trunk, neck, and/or eye(s). The example opening 546 shown in FIG. 5 is elliptical. As shown in FIGS. 5-7, the opening 546 extends through a nozzle wall 548 of the nozzle 538 from an interior nozzle surface 550 of the nozzle wall 548 to an exterior nozzle surface 552 of the nozzle wall 548. The opening 546 is defined by an inner opening surface 570 of the nozzle 538 that extends between the interior and exterior nozzle surfaces 550, 552. The exterior nozzle surface 552 is configured to be directed substantially toward the target site of the user during use. The interior nozzle surface 550, being arranged at an opposite direction from the exterior nozzle surface 552, thus is configured to be directed substantially away from the target site of the user during use.

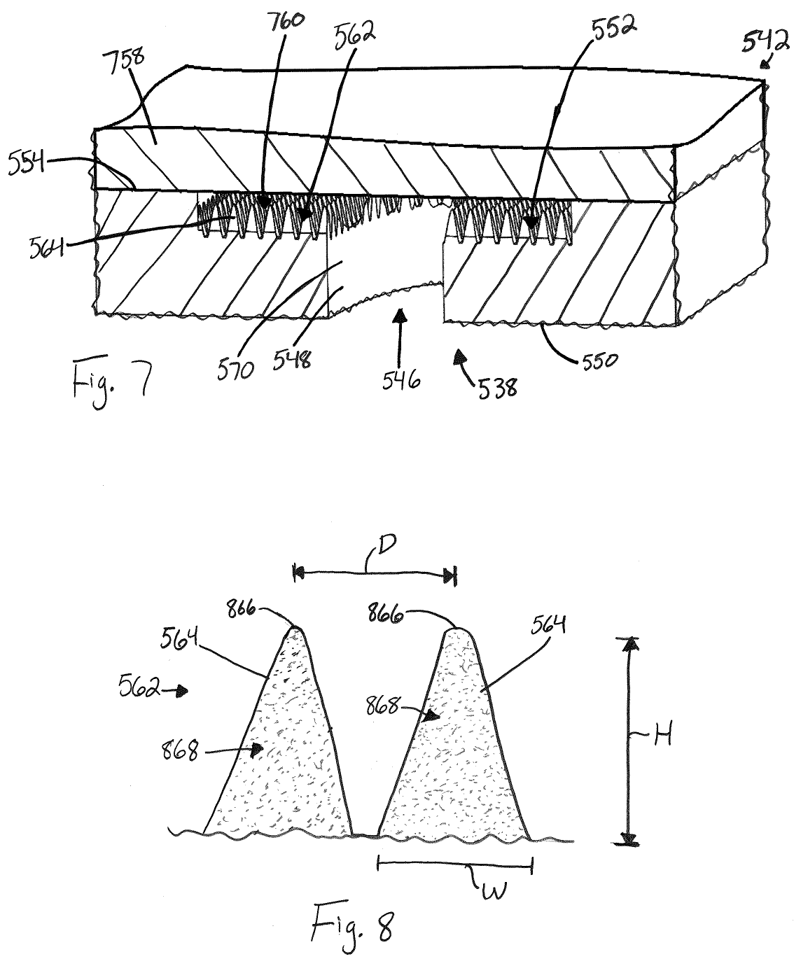

The head 542 may include a head engagement surface 554 that at least partially surrounds the nozzle 538. The head engagement surface 554 may be spaced from the exterior nozzle surface 552 such that an interior head surface 556 extends between the head engagement surface 554 and the exterior nozzle surface 552. As shown in FIG. 7, a cap 758 of the head 542, when in a closed position, may abut the head engagement surface 554. In the closed position, the cap 758 is spaced from the nozzle 538 to form a moisture chamber 760 between the exterior nozzle surface 552 and the cap 758. Spacing the cap 758 from the nozzle 538 in the closed position may help reduce the likelihood of contaminating the nozzle 538 with the cap 758 as the nozzle 538 is not directly touched by the cap 758. Even with this spacing, though, it may be possible for excess fluid and/or outside contaminates (e.g., dust and/or other small particles) to collect on the exterior nozzle surface 552 during use of the fluid delivery device 540.

In order to help prevent the collection of any excess fluid/outside contaminates, and/or help remove such fluid/contaminates when on the exterior nozzle surface, the exterior nozzle surface 552 is configured to be at least hydrophobic or, in some configurations, superhydrophobic. In other words, the exterior nozzle surface 552 is configured such that an effective contact angle of a liquid droplet (e.g., a droplet of the fluid dispensed through the nozzle 538) on the exterior nozzle surface 552 is greater than 90 degrees, e.g., 120 degrees. The hydrophobic exterior nozzle surface 552 may also be configured such that a sliding angle of a liquid droplet (e.g., a droplet of the fluid dispensed through the nozzle 538) on the exterior contact surface 552 is less than 45 degrees. In certain configurations, it may be beneficial for the hydrophobicity of the exterior nozzle surface 552 to be at a superhydrophobic level. The superhydrophobic exterior nozzle surface 552 may be configured such that the effective contact angle is at least 150 degrees. The superhydrophobic exterior nozzle surface 552 may also be configured such that the sliding angle is at most 45 degrees, and preferably below 25 degrees. The exterior nozzle surface 552 can be made hydrophobic (and, when desired, superhydrophobic) through the use of a variety of different hydrophobic mechanisms and combinations thereof.

A first hydrophobic mechanism includes a micropattern 562 that is etched into, formed into, formed with, and/or provided on the exterior nozzle surface 552. FIGS. 5-8 depict one example micropatterned exterior nozzle surface 552. The micropattern 562 includes a plurality of protrusions 564. The protrusions 564 may be arranged in a substantially sinusoidal pattern. The plurality of protrusions 564 provide hydrophobic or superhydrophobic properties to the exterior nozzle surface 552. Each of the protrusions 564 may have a height H of from about 5 to about 25 micrometers and a width W from about 5 to about 25 micrometers. A distance D between the peaks or free ends 866 of adjacent protrusions 564 may be from about 5 to about 50 micrometers.

Although the micropattern 562 is shown as being a sinusoidal micropattern 562, the micropattern 562 may be configured in any other manner in order to provide hydrophobic or superhydrophobic properties. For example, the micropattern 562 may exhibit a variety of geometries (e.g., pillars, channels, platelets, cones, divots, etc.). The micropattern 562 can be substantially constant (e.g., exhibiting a single, repeating feature of substantially unchanging dimensions) and/or can exhibit a substantially repeating pattern (e.g., a plurality of features differing in one or more of size, shape, and spacing, that define an ordered, repeating pattern). The micropattern 562 may be defined at least in part in relation to the size and/or spacing of the geometric elements forming the micropattern 562. The micropattern 562 may be configured to mimic the surface topography of certain surfaces of natural organisms that provide hydrophobic or superhydrophobic properties, such as, for example, a lotus leaf and sharkskin. In the case of a lotus leaf micropattern, the plurality of protrusions 564 may at least partially mimic the size, shape, and/or spacing of the papillae of a lotus leaf. The exterior nozzle surface 552 including a lotus leaf micropattern may thus embody hydrophobic or superhydrophobic properties resembling those of a natural lotus leaf. Additional description with respect to surfaces including a lotus leaf micropattern is provided in Latthe et al. “Superhydrophobic Surfaces Developed by Mimicking Hierarchical Surface Morphology of Lotus Leaf,” Molecules 19 (2014) 4256-83, the subject matter of which is incorporated by reference in its entirety.

As shown in FIG. 7, in the closed position, the cap 758 is spaced from the protrusions 564. Spacing the cap 758 from the protrusions 564 in the closed position helps reduce the likelihood of contaminating the protrusions 564 and/or damaging the protrusions 564 with the cap 758 as the protrusions 564 may be spaced apart from an interior of the cap 758.

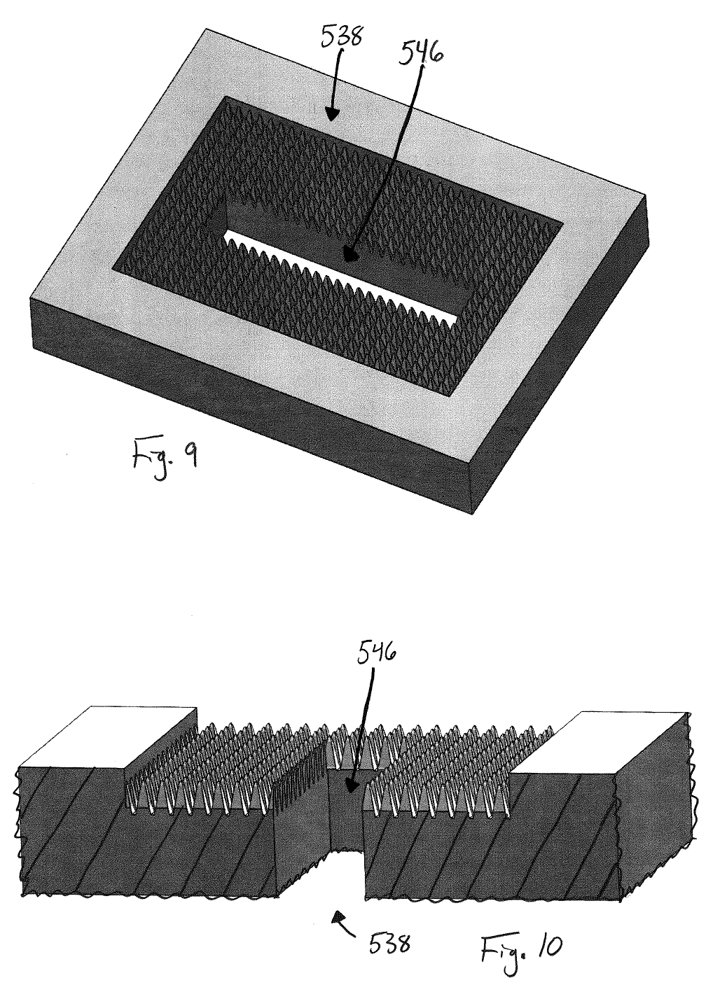

As shown in FIG. 8, a second hydrophobic mechanism includes adding nanometer-sized features 868 to the exterior nozzle surface 552. The nanometer-sized features 868 add nano-scale roughness to the exterior nozzle surface 552 that may at least partially provide hydrophobic or superhydrophobic properties to the exterior nozzle surface 552. The nanometer-sized features 868 may have a height of, for example, about 100 to about 200 nanometers. When the protrusions 564 are provided, the nano-sized features 868 may at least partially add nano-scale roughness to the protrusions 564.

A third hydrophobic mechanism includes applying one or more coatings of a hydrophobic or superhydrophobic material (e.g., non-polar polymers, fluorinated polymers, non-polar siloxanes or silicone-like materials, etc.) to the exterior nozzle surface 552. The coating(s) may be, for example, covalently bonded to the exterior nozzle surface 552 so as to chemically link the coating(s) to the exterior nozzle surface 552 and increase the strength of the attachment between the coating(s) and the exterior nozzle surface 552. The coatings, when applied, manipulates the effective contact angle of the exterior nozzle surface 552 so that the exterior nozzle surface is at least hydrophobic (e.g., in some configurations, superhydrophobic).

When the protrusions 564 are provided, the protrusions 564 may be at least partially coated with the hydrophobic or superhydrophobic material. Similarly, when the nano-sized features 868 are provided, the nano-sized features 868 may be at least partially coated with the hydrophobic or superhydrophobic material. The coating(s), in certain applications, may naturally produce nano-scale roughness on the exterior nozzle surface 552. Therefore, if desired, the use of separate nanometer-sized features 868 may be omitted when such nano-scale roughness is produced via the coating(s).

A fourth hydrophobic mechanism includes configuring a nozzle material that forms the exterior nozzle surface 552 to be at least hydrophobic (e.g., in some configurations, to be superhydrophobic). To configure the nozzle material as such, the nozzle material may be selected or chemically modified such that some portion or all of the exposed groups at the exterior nozzle surface 552 are non-polar, fluorinated, or otherwise intrinsically have hydrophobic or superhydrophobic properties. For example, the nozzle material forming the exterior nozzle surface 552 may be a naturally hydrophobic or naturally superhydrophobic material. The chemical modification of the exterior nozzle surface 552 can occur prior to use of the fluid delivery device 540. When the exterior nozzle surface 552 is chemically modified, the effective contact angle of the exterior nozzle surface 552 is manipulated so that the exterior nozzle surface is at least hydrophobic. When the protrusions 564 are provided, the protrusions 564 may also be configured to have hydrophobic or superhydrophobic properties via the nozzle material if the protrusions 564 are formed from the same nozzle material as the exterior nozzle surface 552 and/or formed integrally or monolithically as one-piece with the exterior nozzle surface 552. Similarly, when the nano-sized features 868 are provided, the nano-sized features 868 may also be configured to have hydrophobic or superhydrophobic properties via the nozzle material if the nano-sized features 868 are formed from the same nozzle material as the exterior nozzle surface 552 and/or formed integrally or monolithically as one-piece with the exterior nozzle surface 552.

Any of the hydrophobic mechanisms disclosed herein could be used, singly, in combination with each other, or in combination with any suitable hydrophobic/superhydrophobic mechanisms, to achieve a desired hydrophobicity on the exterior nozzle surface 552. Furthermore, any of the hydrophobic mechanisms disclosed herein could be used, singly, in combination with each other, or in combination with any suitable hydrophobic/superhydrophobic mechanisms, to achieve superhydrophobicity on the exterior nozzle surface 552. As just one example, the exterior nozzle surface 552 may be configured as superhydrophobic via a combination of the first hydrophobic mechanism (the micropatterning 562) and the third hydrophobic mechanism (the hydrophobic material coating).

The hydrophobic exterior nozzle surface 552 (which may be superhydrophobic in certain configurations) is beneficial in that it may at least partially exhibit a “self-cleaning” behavior. For example, during use of the fluid delivery device 540, extraneous fluid droplets may “roll off” the hydrophobic exterior nozzle surface 552 (via, e.g., gravity or capillarity) and may carry outside contaminants that are on the exterior nozzle surface 552 with them as they “roll off” the exterior nozzle surface 552. Therefore, by being hydrophobic, the exterior nozzle surface 552 may “self-clean” by at least partially preventing the collection of any excess fluid, which may have remained on the exterior nozzle surface 552 had it not been hydrophobic, on the exterior nozzle surface 552. The hydrophobic exterior nozzle surface 552 may also “self-clean” by utilizing the fluid droplets that “roll off” the exterior nozzle surface 552 to remove at least some of the outside contaminates, which may have remained on the exterior nozzle surface 552 had it not been hydrophobic, from the exterior nozzle surface 552.

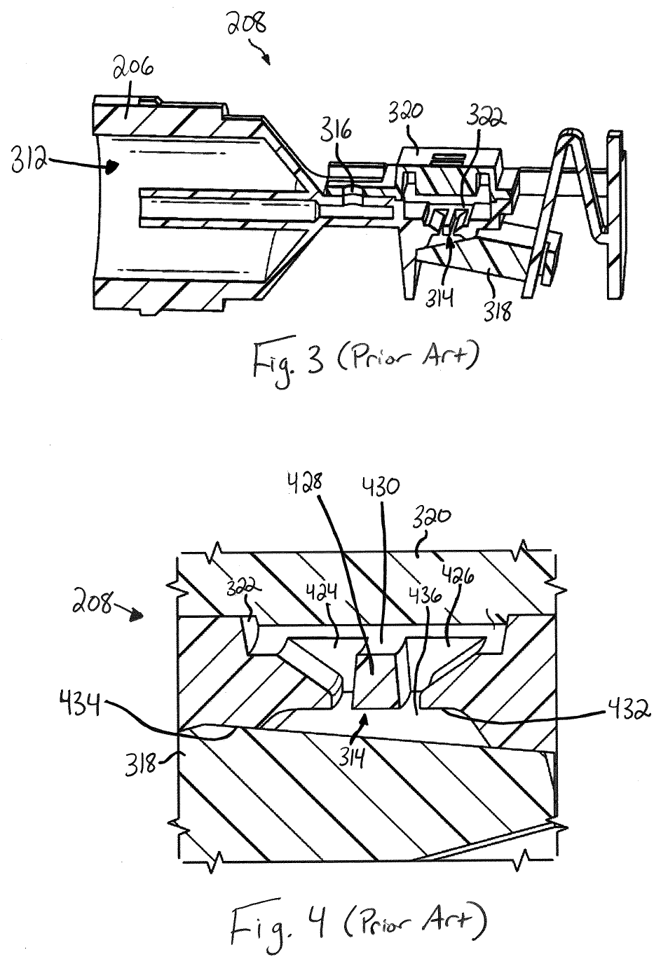

The hydrophobic exterior nozzle surface 552 is also beneficial in that it may at least partially help retain the fluid within a holding chamber (such as, for example, the holding chamber 322 of FIGS. 3-4) against a pressure gradient. Because the exterior nozzle surface 552 is at least hydrophobic (and, thus the contact angle being the exterior nozzle surface 552 and the fluid is at least 90 degrees—and at least 150 when superhydrophobic), a Laplace pressure between the holding chamber and an exterior of the nozzle 538 adjacent the exterior nozzle surface 552 at least partially overcomes forces that may urge the fluid in the holding chamber from undesirably leaking out through the nozzle 538. An example of a force that may be overcome by the Laplace pressure is a hydrostatic pressure of the fluid in the holding chamber.

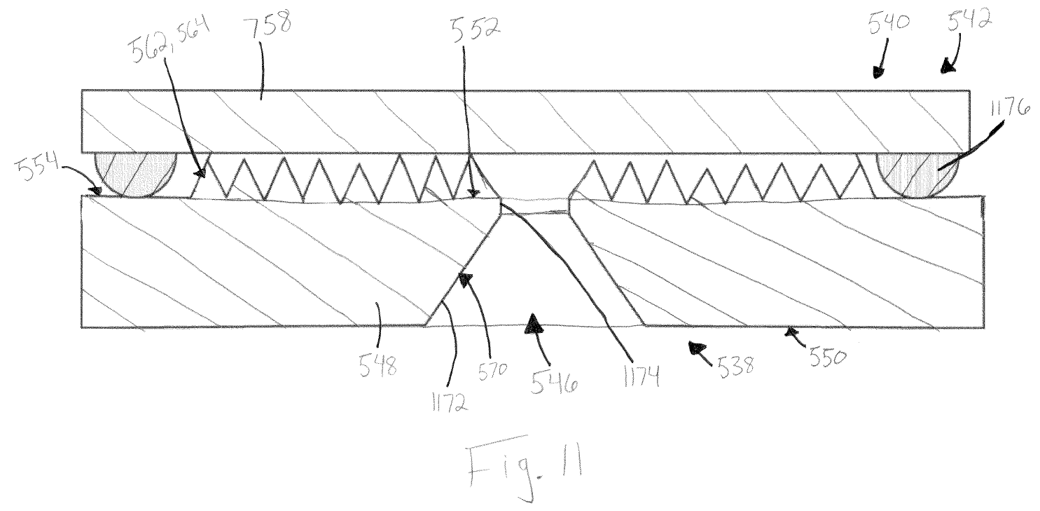

Although the nozzle 538 is shown as having a single elliptical opening 546, the nozzle 538 may have any number of openings with each opening having any desired shape. For example, as disclosed in the '482 application, the nozzle 538 may have an array of openings (e.g., conically-shaped openings), a singular stadium-shaped opening, a singular undulating stadium-shaped opening, a singular “bow-tie” shaped opening, as singular S-shaped opening, and/or two slit openings. As shown in FIGS. 9-10, the opening 546 could also be rectangular.

The micropatterns and topology discussed and depicted in the present disclosure may be practically made and reproduced at low cost using plastic microinjection molding. For example, a low surface energy polypropylene or high density polyethylene (HDPE) material, which are common materials used to make ophthalmic eye drop containers, may be used in conjunction with micromolding techniques to make the geometries and hydrophobic/superhydrophobic surface patterns discussed herein. Opposing sides of the nozzles 538 may each be defined by an external hard steel tooling for the micromolding process. The external hard steel tooling may be joined to one another to form a molding cavity in the shape of the nozzles 538. Appropriate angular draft may be needed on all features so that the tooling can easily separate after plastic injection.

Although only the exterior nozzle surface 552 has been shown as being hydrophobic or superhydrophobic, any other surface of the fluid delivery device 540 may also or instead be configured to be hydrophobic/superhydrophobic in a similar manner as described above. For example, at least one of the head engagement surface 554 and the interior head surface 556 may be at least hydrophobic. In on particular example, the interior head surface 556 may include the nanometer-sized features 868 that add nano-scale roughness to the interior head surface 556. Any hydrophobic/superhydrophobic surface may at least provide the same “self-cleaning” and/or fluid-retention benefits as described above.

It is contemplated that while at least one surface of the fluid delivery 540 device is superhydrophobic, at least one other surface of the fluid delivery device 540 may be configured to be hydrophobic via one or more of the hydrophobic mechanisms described above and/or via other known hydrophobic mechanisms that provides hydrophobic properties. A hydrophobic surface has an effective contact angle between the hydrophobic surface and a liquid droplet (e.g., a droplet of the fluid dispensed through the nozzle 538) placed over the hydrophobic surface of at least 90 degrees. As an example, the exterior nozzle surface 552 may be superhydrophobic, while the interior head surface 556 may be hydrophobic.

While at least the exterior nozzle surface 552 is described as being at least hydrophobic, at least one “interior” surface of the nozzle 538 and/or head 542 may be hydrophilic. A hydrophilic surface has a less than 90 degree effective contact angle between the hydrophilic surface and a liquid droplet placed over the hydrophilic surface. For example, at least one of the interior nozzle surface 550 and the inner opening surface 570 may be configured to be hydrophilic. The exterior nozzle surface 552 can be made interior through the use of a variety of different hydrophilic mechanisms and combinations thereof.

A first hydrophilic mechanism includes applying one or more coatings of a hydrophilic material (e.g., oxides, silica-dioxide(s), glass-like PECVD coatings, etc.) to at least one of the interior nozzle surface 550 and the inner opening surface 570. The hydrophilic coating(s) may be, for example, covalently bonded to its respective surface(s) so as to chemically link the coating(s) to its respective surface(s) and increase the strength of the attachment between the coating(s) its respective surface(s). The coatings, when applied, manipulates the effective contact angle of at least one of the interior nozzle surface 550 and the inner opening surface 570 so that at least one of the interior nozzle surface 550 and the inner opening surface 570 is hydrophilic.

A second hydrophilic mechanism includes configuring a nozzle material that forms at least one of the interior nozzle surface 550 and the inner opening surface 570 to be hydrophilic. To configure the nozzle material as such, the nozzle material forming at least one of the interior nozzle surface 550 and the inner opening surface 570 may be selected or chemically modified such that some portion or all of the exposed groups of at least one of the interior nozzle surface 550 and the inner opening surface 570 have hydrophilic properties. For example, the nozzle material forming at least one of the interior nozzle surface 550 and the inner opening surface 570 may be a naturally hydrophilic material (e.g., a hydrophilic plastic such as some grades of (PET) or polyethylene terephthalate). The chemical modification of at least one of the interior nozzle surface 550 and the inner opening surface 570 can occur prior to use of the fluid delivery device 540. When at least one of the interior nozzle surface 550 and the inner opening surface 570 is chemically modified, the effective contact angle of at least one of the interior nozzle surface 550 and the inner opening surface 570 is manipulated so that the exterior nozzle surface is hydrophilic.

The hydrophilic mechanisms of coating and/or chemically modifying at least one of the interior nozzle surface 550 and the inner opening surface 570 so as to be hydrophilic is particularly useful when at least one of the interior nozzle surface 550 and the inner opening surface 570 is formed from a naturally hydrophobic material (e.g., polypropylene).

Any of the hydrophilic mechanisms disclosed herein could be used, singly, in combination with each other, or in combination with any suitable hydrophilic mechanisms, to achieve a desired hydrophilicity on at least one of the interior nozzle surface 550 and the inner opening surface 570. Ideally, each of the interior surfaces of the holding chamber and/or the nozzle 538 (including the interior nozzle surface 550 and the inner opening surface 570) are hydrophilic, as this configuration helps resist the formation of air bubbles (which could impede the ejection of the fluid) inside the interior of the nozzle 538 and/or the holding chamber by making it more energetically favorable for aqueous solutions to wet all the surfaces of such interior surfaces. However, it is possible to achieve the same, similar, or at least some air bubble-formation resistance even when at least one of the interior nozzle surface 550 and the inner opening surface 570 are at least partially hydrophilic.

In certain configurations of the nozzle 538, less than an entire extent of the inner opening surface 570 from the interior nozzle surface 550 to the exterior nozzle surface 552 is hydrophilic. In such configurations, the inner opening surface 570 may include a transition portion directly between a hydrophilic portion and the exterior nozzle surface. This transition portion thus is adjacent to the exterior nozzle surface 552 and spaced from the interior nozzle surface 550. FIG. 11 illustrates one example of such a nozzle 538 configuration.

The opening 546 of the nozzle 538 of FIG. 11 at least partially inwardly tapers as it extends from the interior nozzle surface 550 to the exterior nozzle surface 552. The inner opening surface 570 thus includes a sloped portion 1172 and a transition portion 1174 that extends directly in a fluid ejection direction from the sloped portion 1172 to the exterior nozzle surface 552. In the configuration of FIG. 11, the sloped portion 1172 is configured to be hydrophilic via any one or more of the hydrophilic mechanisms described above and/or any other suitable hydrophilic mechanism, while the transition portion 1174 is configured to be at least hydrophobic via any one or more of the hydrophobic mechanisms described above and/or any other suitable hydrophobic mechanism. A length of the transition portion 1174, which is measured in the fluid ejection direction, may be less than (e.g., significantly less than) a width of the opening 546, measured in a direction perpendicular to the fluid ejection direction, at the transition portion 1174.

The hydrophobicity of the transition portion 1174 may be the same as or different than the hydrophobicity of the exterior nozzle surface 552. For example, the transition portion 1174 may have a lower hydrophobicity than that of the exterior nozzle surface 552 so that the hydrophobicity more smoothly transitions from the hydrophilic sloped portion 1172 to the hydrophobic exterior nozzle surface 552. As another example, the transition portion 1174 may be hydrophobic and the exterior nozzle surface 552 may be superhydrophobic.

Although the head engagement surface 554 was previously described as being spaced from the exterior nozzle surface 552 by the interior head surface, the head engagement surface 554 may form a single continuous surface with the exterior nozzle surface 552. In such case, the cap 758 may include at least one projection that extends toward and contacts the head engagement surface 554 when the cap 758 is in the closed position. The at least one projection may be integrally formed with the cap 758 as a single piece or may be separately formed and subsequently attached to the cap 758. The separately formed projection may be formed from an elastic material and/or may be in the form of an O-ring. An example configuration of the cap 758 having a separately formed elastic projection 1176 and the head engagement surface 554 forming a single continuous surface with the exterior nozzle surface 552 is illustrated in FIG. 11.

While aspects of this disclosure have been particularly shown and described with reference to the example aspects above, it will be understood by those of ordinary skill in the art that various additional aspects may be contemplated. For example, the specific methods described above for using the apparatus are merely illustrative; one of ordinary skill in the art could readily determine any number of tools, sequences of steps, or other means/options for placing the above-described apparatus, or components thereof, into positions substantively similar to those shown and described herein. In an effort to maintain clarity in the Figures, certain ones of duplicative components shown have not been specifically numbered, but one of ordinary skill in the art will realize, based upon the components that were numbered, the element numbers which should be associated with the unnumbered components; no differentiation between similar components is intended or implied solely by the presence or absence of an element number in the Figures. Any of the described structures and components could be integrally formed as a single unitary or monolithic piece or made up of separate sub-components, with either of these formations involving any suitable stock or bespoke components and/or any suitable material or combinations of materials. Any of the described structures and components could be disposable or reusable as desired for a particular use environment. Any component could be provided with a user-perceptible marking to indicate a material, configuration, at least one dimension, or the like pertaining to that component, the user-perceptible marking potentially aiding a user in selecting one component from an array of similar components for a particular use environment. The term “substantially” is used herein to indicate a quality that is largely, but not necessarily wholly, that which is specified—a “substantial” quality admits of the potential for some relatively minor inclusion of a non-quality item. Though certain components described herein are shown as having specific geometric shapes, all structures of this disclosure may have any suitable shapes, sizes, configurations, relative relationships, cross-sectional areas, or any other physical characteristics as desirable for a particular application. Any structures or features described with reference to one aspect or configuration could be provided, singly or in combination with other structures or features, to any other aspect or configuration, as it would be impractical to describe each of the aspects and configurations discussed herein as having all of the options discussed with respect to all of the other aspects and configurations. A device or method incorporating any of these features should be understood to fall under the scope of this disclosure as determined based upon the claims below and any equivalents thereof.

Other aspects, objects, and advantages may be obtained from a study of the drawings, the disclosure, and the appended claims.

We claim:

1. A nozzle for an ophthalmic fluid delivery device, the nozzle comprising:
a nozzle wall having opposing interior and exterior nozzle surfaces, and at least one opening through which fluid is configured to be selectively delivered to an eye of a user during use of the ophthalmic fluid delivery device, the at least one opening being defined by an inner opening surface and extending through the nozzle wall from the interior nozzle surface to the exterior nozzle surface, the exterior nozzle surface being configured to be directed toward the eye of the user during use of the fluid delivery device, the exterior nozzle surface being configured to be at least hydrophobic, at least a portion of the inner opening surface being configured to be hydrophilic.

2. The nozzle of claim 1, wherein the exterior nozzle surface is configured to be at least hydrophobic via at least one of a micropattern or nano-sized features on the exterior nozzle surface.

3. The nozzle of claim 2, wherein the micropattern on the exterior nozzle surface is defined by a plurality of micro-sized protrusions.

4. The nozzle of claim 1, wherein the exterior nozzle surface is superhydrophobic with a less than 45 degree sliding angle for a liquid droplet on the exterior contact surface.

5. The nozzle of claim 1, wherein the exterior nozzle surface is configured to be at least hydrophobic via forming the exterior nozzle surface from a material that is at least hydrophobic.

6. The nozzle of claim 1, wherein the exterior nozzle surface is configured to be at least hydrophobic via a hydrophobic or superhydrophobic coating on the exterior nozzle surface.

7. The nozzle of claim 6, wherein the coating is covalently bonded to the exterior nozzle surface.

8. The nozzle of claim 6, wherein the coating may be composed of at least one of non-polar polymers, fluorinated polymers, and silicone materials.

9. The nozzle of claim 1, wherein the interior nozzle surface is configured to be hydrophilic.

10. The nozzle of claim 1, wherein the hydrophilic portion of the inner opening surface is configured to be hydrophilic via a hydrophilic coating.

11. The nozzle of claim 10, wherein the hydrophilic coating is covalently bonded to the inner opening surface.

12. The nozzle of claim 1, wherein the hydrophilic portion of the inner opening surface is configured to be hydrophilic via forming the hydrophilic portion of the inner opening surface from a hydrophilic material.

13. The nozzle of claim 1, wherein the hydrophilic portion of the inner opening surface is configured to be hydrophilic via a chemical modification.

14. The nozzle of claim 1, wherein the ophthalmic fluid delivery device is a non-gravitational ophthalmic delivery device for delivering fluids having aqueous-based formulations.

15. The nozzle of claim 1, wherein the inner opening surface includes a hydrophobic portion and a hydrophilic portion, the hydrophobic portion being between the hydrophobic portion and the exterior nozzle surface.

16. A nozzle for an ophthalmic fluid delivery device, the nozzle comprising:
a nozzle wall having opposing interior and exterior nozzle surfaces, and at least one opening through which fluid is configured to be selectively delivered to an eye of a user during use of the ophthalmic fluid delivery device, the at least one opening being defined by an inner opening surface and extending through the nozzle wall from the interior nozzle surface to the exterior nozzle surface, the exterior nozzle surface being configured to be directed toward the eye of the user during use of the fluid delivery device;
wherein the exterior nozzle surface is at least hydrophobic via at least one of
a micropattern on the exterior nozzle surface;
a hydrophobic or superhydrophobic coating on the exterior nozzle surface;
a material forming the exterior nozzle surface being naturally hydrophobic or superhydrophobic;
a chemical modification of the exterior nozzle surface; and
nanometer-sized features on the exterior nozzle surface; and
wherein at least one of the interior nozzle surface and the inner opening surface is configured to be hydrophilic via at least one of
a hydrophilic coating on at least one of the interior nozzle surface and the inner opening surface;
a material forming at least one of the interior nozzle surface and the inner opening surface being naturally hydrophilic; and
a chemical modification of the exterior nozzle surface.

17. The nozzle of claim 16, wherein the exterior nozzle surface is superhydrophobic with a less than 45 degree sliding angle for a liquid droplet on the exterior contact surface.

18. The nozzle of claim 16, wherein the exterior nozzle surface is at least hydrophobic via the hydrophobic or superhydrophobic coating on the exterior nozzle surface, the hydrophobic or superhydrophobic coating being covalently bonded to the exterior nozzle surface.

19. The nozzle of claim 16, wherein at least one of the interior nozzle surface and the inner opening surface is configured to be hydrophilic via the hydrophilic coating on at least one of the interior nozzle surface and the inner opening surface, the hydrophilic coating being covalently bonded to at least one of the interior nozzle surface and the inner opening surface.

20. The nozzle of claim 16, wherein each of the interior nozzle surface and the inner opening surface are configured to be hydrophilic.

21. The nozzle of claim 16, wherein the ophthalmic fluid delivery device is a non-gravitational ophthalmic delivery device for delivering fluids having aqueous-based formulations.

22. The nozzle of claim 16, wherein the inner opening surface includes a hydrophobic portion and a hydrophilic portion, the hydrophobic portion being between the hydrophobic portion and the exterior nozzle surface.
